(19) United States
(12) Patent Application Publication
(10) Pub. No.: US 2025/0120918 A1
(43) Pub. Date: Apr. 17, 2025

(54) NEW FORMULATIONS OF PITOLISANT AND METHODS OF USE

(71) Applicant: Bioprojet Pharma, Paris (FR)

(72) Inventors: Marc CAPET, Melesse (FR); Philippe ROBERT, Grenoble (FR); Olivier FINANCE, Rennes (FR); Jeanne-Marie LECOMTE, Paris (FR); Jean-Charles SCHWARTZ, Paris (FR); Elsie MELSOPP, Wilmington, NC (US); Kumaraswamy BUDUR, Chicago, IL (US); Jean-Stéphane JULIEN, Saint-Cyr-sur-Loire (FR)

(21) Appl. No.: 18/917,157

(22) Filed: Oct. 16, 2024

Related U.S. Application Data
(60) Provisional application No. 63/590,678, filed on Oct. 16, 2023.
(60) Provisional application No. 63/616,039, filed on Dec. 29, 2023.
(60) Provisional application No. 63/679,270, filed on Aug. 5, 2024.

(30) Foreign Application Priority Data
Jul. 18, 2024 (EP) 24306216.3

(51) Int. Cl. A61K 9/20; A61K 9/28; A61K 31/335; A61K 31/4453

(52) U.S. Cl. CPC A61K 9/2009; A61K 9/2018; A61K 9/2054; A61K 9/28; A61K 31/335; A61K 31/4453

(57) ABSTRACT

The present disclosure relates to combinations, dosage forms, and pharmaceutical formulations that comprise an active pharmaceutical ingredient, such as pitolisant, and an alkalinizing agent for improving absorption of the active pharmaceutical ingredient.

### RELATED APPLICATIONS

This application claims the benefit of U.S. Provisional Application No. 63/590,678, filed on Oct. 16, 2023, U.S. Provisional Application No. 63/616,039, filed on Dec. 29, 2023, U.S. Provisional Application No. 63/679,270, filed on Aug. 5, 2024, and European Patent Application No. 24306216.3, filed on Jul. 18, 2024, the entire contents of which are each incorporated herein by reference.

### BACKGROUND

Pitolisant hydrochloride is a drug molecule that is useful for treating various diseases and disorders, particularly sleep disorders such as excessive daytime sleepiness (EDS) and cataplexy, that is marketed as WAKIX®. WAKIX® contains crystalline pitolisant hydrochloride. Pitolisant is typically administered orally and absorbed with a peak plasma concentration (Cₘₐₓ) reached approximately three hours after oral administration and the plasmatic elimination half-life of pitolisant is 8 to 11 hours.

In order to achieve its awakening effect without affecting the nocturnal sleep of the patient, it is desirable that the effect of pitolisant is triggered as soon as possible after administration. It is thus desirable to shorten the absorption time of pitolisant.

Doxepin hydrochloride is another drug molecule, which was developed as an antidepressant but has also been approved in the treatment of insomnia. Doxepin is typically administered orally, and the peak plasma concentration of doxepin is reached 3.5 hours after oral administration. A quick onset of the effect of doxepin is desired in order to promote sleep.

There is a recognized but unmet need for shortening the time of absorption of active pharmaceutical ingredients, in particular active ingredients used for treating sleep disorders or diseases, and particularly active pharmaceutical ingredients that are administered orally.

### SUMMARY

The present disclosure relates generally to combinations of an active pharmaceutical ingredient (API) and an alkalinizing agent, as well as dosage forms and pharmaceutical compositions comprising the same. The combinations, dosage forms, and pharmaceutical compositions of the present disclosure can provide a more rapid absorption of the API, e.g., following oral administration, compared to when administering the API without the combination or as another dosage form or pharmaceutical composition.

It has now been discovered that active pharmaceutical ingredients that are typically administered in the form of a chloride salt may be more rapidly absorbed if they are administered in combination with an alkalinizing agent.

Without wishing to be bound by theory, it is believed that APIs that are typically administered in the form of a salt, such as a hydrochloride salt (e.g., pitolisant hydrochloride or doxepin hydrochloride) are more rapidly absorbed if they are administered in combination with an alkalinizing agent, e.g., following oral administration.

In particular, it has been shown that the plasmatic absorption peak of pitolisant hydrochloride or doxepin hydrochloride occurs more rapidly when co-administered with an alkalinizing agent.

For example, it is believed that the plasmatic absorption peak (or Cₘₐₓ) of pitolisant hydrochloride or doxepin hydrochloride occurs more rapidly when co-administered with an alkalinizing agent (i.e., the Tₘₐₓ is shortened).

The present disclosure generally relates to a new use allowing, inter alia, accelerated crossing of an active pharmaceutical ingredient through the intestinal barrier.

More specifically, the present disclosure also generally relates to dosage forms and pharmaceutical compositions comprising the combination of an active pharmaceutical ingredient and an alkalinizing agent, and uses thereof, resulting in, inter alia, accelerated absorption or crossing of the active pharmaceutical ingredient (e.g., from the gastrointestinal tract) e.g., through the intestinal barrier and into the bloodstream, e.g., following oral administration.

In particular, the present disclosure relates to a combination of a salt of an active pharmaceutical ingredient with an alkalinizing agent. More specifically, the present disclosure relates to a combination of a salt of an active pharmaceutical ingredient and an alkalinizing agent, as well as dosage forms and pharmaceutical compositions comprising the same.

The present disclosure also relates to new formulations of API, such as pitolisant (e.g., pitolisant hydrochloride), with improved absorption of the API (e.g., following oral administration of the formulation).

In an embodiment, the present disclosure relates to an accelerated absorption formulation comprising an active pharmaceutical ingredient, in the form of a salt, together with an alkalinizing agent.

In an embodiment, the present disclosure relates to the use of an alkalinizing agent to accelerate the absorption of an active pharmaceutical ingredient administered in the form of a salt.

In some embodiments, the salt is a hydrochloride salt.

In some embodiments, active pharmaceutical ingredients administered in the form of a salt, such as hydrochloride, particularly relate to pitolisant or doxepin.

The present disclosure also relates to a combination of a pitolisant salt, such as pitolisant hydrochloride, with an alkalinizing agent.

The present disclosure also relates to a pharmaceutical composition or dosage form comprising the combination of salt of pitolisant, such as pitolisant hydrochloride, an alkalinizing agent as defined herein, and optionally one or more pharmaceutically acceptable excipients.

### BRIEF DESCRIPTION OF DRAWINGS

FIG. 1 is a graphical representation of the cumulative apparent permeability (Papp) (10⁶ cm/s) over time (0-120 min) for exemplary combinations of pitolisant and an alkalinizing agent in different amounts, as compared with control.

FIG. 2 is a graphical representation that illustrates the cumulative apparent permeability expressed as bioavailability (area under the curve, AUC₀₋₁₂₀ mi) (expressed as % over control) for exemplary combinations of pitolisant hydrochloride having alkalinizing agents in different amounts (numbers 2-13) as compared to control (number 1).

FIG. 3 is a graphical representation that illustrates the apparent permeability (Papp) (10⁶ cm/s) of doxepin hydrochloride over time (120 min) using an exemplary combination of doxepin hydrochloride, compared to a control.

### DETAILED DESCRIPTION

The present disclosure relates to combinations of an active pharmaceutical ingredient (API), which can be in the form of a pharmaceutically acceptable salt, such as a hydrochloride salt, and an alkalinizing agent. In some embodiments, the disclosure relates to combinations of an active pharmaceutical ingredient in the form of a pharmaceutically acceptable salt, preferably hydrochloride salt, together with an alkalinizing agent.

More specifically, the present disclosure relates to combinations of pitolisant in the form of a pharmaceutically acceptable salt, preferably a hydrochloride salt, with an alkalinizing agent.

The present disclosure also relates to combinations of doxepin in the form of a pharmaceutically acceptable salt, preferably hydrochloride salt, with an alkalinizing agent.

The present disclosure further relates to dosage forms and pharmaceutical compositions comprising a combination of the present disclosure, e.g., a combination of pitolisant hydrochloride and an alkalinizing agent.

The present disclosure also relates to the use of the combinations, dosage forms, and pharmaceutical compositions of the present disclosure, such as to treat a disease or disorder in a subject in need thereof.

### Active Pharmaceutical Ingredients

The active pharmaceutical ingredient generally refers to the component (e.g., compound, or a pharmaceutically acceptable salt thereof) of a combination, dosage form, or pharmaceutical composition of the present disclosure that can provide a therapeutic effect. The active pharmaceutical ingredient may be referred to herein as API, active ingredient, or drug.

Preferred active pharmaceutical ingredients of the present disclosure are compounds that are formulated in the form of an acid addition salt, such as a hydrochloric acid addition salt. Hydrochloric acid addition salts may be referred to herein as a hydrochloride salts or HCl salts.

In an embodiment, the active pharmaceutical ingredient is pitolisant, or more preferably, an acid addition salt of pitolisant, e.g., pitolisant hydrochloride.

A hydrochloric acid addition salt of pitolisant has the following formula:

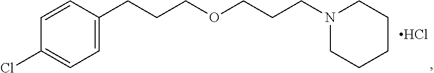 [SMILES: Clc1ccc(CCCOCCCN2CCCCC2)cc1 ; Cl]

which may be referred to herein as pitolisant hydrochloride, pitolisant monohydrochloride, or pitolisant HCl. It will be understood that these terms can encompass amorphous or crystalline forms of pitolisant hydrochloride, such as its polymorphic forms including Form I (disclosed, e.g., in U.S. Pat. No. 8,207,197 that is incorporated herein by reference in its entirety) and its Form II (disclosed in U.S. patent application Ser. No. 18/621,972, which published as U.S. Patent Publication No. 2024/0327366, and PCT/IB2024/000139, which published as WO 2024/201139, that are each incorporated herein by reference in their entireties).

In some embodiments, the active pharmaceutical ingredient is crystalline pitolisant hydrochloride. In some embodiments, the active pharmaceutical ingredient is the Form I polymorph of pitolisant hydrochloride. In some embodiments, the active pharmaceutical ingredient is crystalline pitolisant hydrochloride that has an X-ray diffractogram that comprises characteristic peaks (2θ) at 11.2°, 19.9°, 20.7°, and 34.1° (±0.2°), or more preferably, at 11.2°, 15.4°, 16.3°, 16.9°, 17.8°, 19.9°, 20.7°, 21.0°, 21.8°, 22.6°, 24.5°, 24.6°, 25.0°, 25.5°, 26.3°, 28.3°, 30.3°, 34.1°, 35.8°, 40.0°, and 46.0° (±0.2°). In some embodiments, the active pharmaceutical ingredient is a polymorph of pitolisant as described and characterized in U.S. Pat. No. 8,207,197.

In some embodiments, the active pharmaceutical ingredient is doxepin, or more preferably, an acid addition salt of doxepin, e.g., doxepin hydrochloride.

A hydrochloric acid addition salt of doxepin has the following formula:

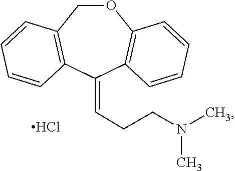 [SMILES: CN(C)CC/C=C1/c2ccccc2COc2ccccc21 ; Cl]

which may be referred to herein as doxepin hydrochloride, doxepin monohydrochloride, or doxepin HCl. It will be understood that these terms can encompass amorphous or crystalline forms of doxepin hydrochloride, such as its polymorphic forms.

The amount of active pharmaceutical ingredient in a combination, dosage form, or pharmaceutical composition of the present disclosure may be expressed as a percentage by weight of the active ingredient in the total weight of the combination, dosage form, or pharmaceutical composition (wt %). For example, a combination, dosage form, or pharmaceutical composition of the present disclosure may comprise an active ingredient in an amount of between about 1 wt % and about 99 wt %, e.g., between about 1 wt % and about 80 wt %, between about 1 wt % and about 60 wt %, between about 1 wt % and about 40 wt %, between about 1 wt % and about 30 wt %, between about 1 wt % and about 20 wt %, between about 1 wt % and about 10 wt %, between about 10 wt % and about 15 wt %, e.g., about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 30 wt %, about 35 wt %, about 40 wt %, about 50 wt %, about 60 wt %, about 70 wt %, about 80 wt %, about 90 wt %, about 95 wt %, about 97.5 wt %, or about 99 wt %.

### Salts

Salts typically refer to pharmaceutically acceptable salts of a compound (e.g., the API), such as acid addition salts. Such salts are generally used to achieve a crystalline and/or solid form of a compound that may be otherwise amorphous and/or liquid.

In some embodiments, salts are hydrochloride salts. Such hydrochloride acid addition salts are typically considered to be pharmaceutically acceptable.

Salts can be obtained by contacting the neutral form of the active pharmaceutical ingredient with an acid (e.g., hydrochloric acid), either neat or in a suitable solvent (e.g., an inert solvent). Procedures used to prepare such salts are known to those of skill in the art.

### Alkalinizing Agent

The combinations, dosage forms, and pharmaceutical compositions of the present disclosure comprise an alkalinizing agent.

An alkalinizing agent is an agent that is able to increase the pH, e.g., of a solution (e.g., an aqueous solution). The alkalinizing agent can be a salt, such as a magnesium, calcium, or sodium salt together with a suitable counter-ion.

According to an embodiment, counter-ions can include carbonate, citrate, bicarbonate, acetate, lactate, or oxide salts.

In embodiments, the alkalinizing agent includes one or more of the following: tetraborate, hydroxide, oxide, carbonate, citrate, bicarbonate, acetate, lactate, arginine, lysine, choline, meglumine, tromethamine, potassium, magnesium, calcium, or sodium salts.

Preferably, said alkalinizing agent is chosen from magnesium carbonate, sodium carbonate, potassium bicarbonate, magnesium oxide, or more preferably, the alkalinizing agent is magnesium carbonate.

The amount of alkalinizing agent in a combination, dosage form, or pharmaceutical composition of the present disclosure may be expressed as a percentage by weight of the alkalinizing agent in the total weight of the combination, dosage form, or pharmaceutical composition (wt %). For example, a combination, dosage form, or pharmaceutical composition of the present disclosure may comprise an alkalinizing agent in an amount of between about 1 wt % and about 99 wt %, e.g., between about 1 wt % and about 80 wt %, between about 1 wt % and about 60 wt %, between about 1 wt % and about 40 wt %, between about 1 wt % and about 30 wt %, between about 1 wt % and about 20 wt %, between about 1 wt % and about 10 wt %, between about 1 wt % and about 5 wt %, between about 5 wt % and about 10 wt %, between about 10 wt % and about 15 wt %, e.g., about 1 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 30 wt %, about 35 wt %, about 40 wt %, about 45 wt %, or about 50 wt %.

The alkalinizing agent can be provided in a combination, dosage form, or pharmaceutical composition of the present disclosure in any desired ratio with other component(s) of the combination, dosage form, or pharmaceutical composition, e.g., the active pharmaceutical ingredient. For example, the weight ratio of alkalinizing agent to active pharmaceutical ingredient combination, dosage form, or pharmaceutical composition of the present disclosure can be about 1:1, about 1:2, about 1:3, about 1:4, about 1:5, about 1:6, about 1:7, about 1:8, about 1:9, about 1:10, about 2:3, about 2:5, about 2:7, about 2:9, about 3:4, about 3:5, about 3:7, about 3:8, about 3:9, about 3:10, about 4:5, about 4:7, about 4:9, about 5:6, about 5:7, about 5:8, about 5:9, about 6:7, about 6:9, about 7:8, about 7:9, about 8:9, in the combination, dose form, or pharmaceutical composition.

In an embodiment, the weight ratio (alkalinizing agent/alkalinizing agent+pitolisant salt or alkalinizing agent/alkalinizing agent+doxepin) is comprised between 5 and 99%, preferably between 15 and 95% more preferably between 15 and 70%.

According to an embodiment, the combination, dosage form, or pharmaceutical composition has a pH comprised between 7.5 and 11. For example, when the combination, dosage form, or pharmaceutical composition is exposed to a solution (e.g., an aqueous solution typically drinking water, such as a standardized volume of 250 ml of drinking water), the pH of the solution will be changed to a value between 7.5 and 11. In some embodiments, this change in pH occurs in the microenvironment of the combination, dosage form, or pharmaceutical composition, e.g., within the gastrointestinal tract of a subject that was orally administered the combination, dosage form, or pharmaceutical composition.

### Pharmacokinetics

The combinations of the present disclosure typically exhibit a more rapid absorption of the active ingredient within the body than the active ingredient. More specifically, the combinations, dosage forms, and pharmaceutical compositions of the present disclosure can provide a more rapid absorption of the active ingredient within the body (e.g., following oral administration), e.g., relative to the active ingredient administered without the combination or in another dosage form or pharmaceutical composition.

In particular, and without wishing to be bound by theory, it is believed that the plasmatic absorption peak of the active ingredient (e.g., pitolisant or doxepin) occurs more rapidly when the active ingredient is co-administered with an alkalinizing agent.

Accordingly, the combinations of the present disclosure allow an accelerated absorption (ie) a decrease of the so-called Tₘₐₓ. More specifically, the combinations, dosage forms, and pharmaceutical compositions of the present disclosure can provide an accelerated absorption and/or a decrease of Tₘₐₓ of the active ingredient (e.g., following oral administration), e.g., relative to when administering the active ingredient without the combination or in another dosage form or pharmaceutical composition. It will be understood that Tₘₐₓ is a measure of the minimum time to reach the peak plasma concentration (Cₘₐₓ) of the active ingredient after administration.

The combinations, dosage forms, and pharmaceutical compositions of the present disclosure can provide an increase in Cₘₐₓ (e.g., following oral administration), e.g., relative to the Cₘₐₓ achieved following administration of the active ingredient without the combination or in another dosage form or pharmaceutical composition. In some embodiments, the Cₘₐₓ following administration of the combination can be more than 5%, preferably more than 10%, still more preferably more than 20% of the Cₘₐₓ obtained with the corresponding active ingredient, e.g., when administered without the combination or as another dosage form or pharmaceutical composition.

In some embodiments, the Tₘₐₓ following administration of the combination can be less than 5%, preferably less than 10%, still more preferably less than 20% of the Tₘₐₓ obtained with the corresponding active ingredient, e.g., when administered without the combination or as another dosage form or pharmaceutical composition.

Without wishing to be bound by theory, It is also believed that the combinations of the present disclosure may allow higher absorption of the active ingredient. In particular, it is believed that the combinations, dosage forms, and pharmaceutical compositions of the present disclosure provide higher absorption of the active ingredient (e.g., following oral administration), e.g., relative to when administering the same active ingredient without the combination or as another dosage form or pharmaceutical composition.

Higher absorption can be determined by a higher integral of the concentration-time curve (an increased AUC). In embodiments, the AUC of the combination may be more than 5%, preferably more than 10%, still more preferably more than 20% of the AUC obtained with the corresponding active ingredient. For example, the AUC resulting from administration (e.g., oral administration) of a combination, dosage form, or pharmaceutical composition of the present disclosure can be more than 5%, preferably more than 10%, still more preferably more than 20% of the AUC obtained with the corresponding active ingredient, e.g., as administered without the combination or as another dosage form or pharmaceutical composition.

### Dosage Forms and Pharmaceutical Compositions

The present disclosure also relates to a dosage form or pharmaceutical composition comprising a combination disclosed herein, and optionally one or more pharmaceutically acceptable excipients.

The present disclosure also relates to a dosage form comprising a pharmaceutical composition disclosed herein.

Dosage forms and pharmaceutical compositions of the present disclosure can comprise a pitolisant salt, such as pitolisant hydrochloride, and an alkalinizing agent, and optionally one or more pharmaceutically acceptable excipients.

A pharmaceutical composition or dosage form of the present disclosure can be a tablet, caplet, capsule, suspension, granule, powder, or the like. A pharmaceutical composition or dosage form of the present disclosure can be in the form of a tablet. A pharmaceutical composition or dosage form of the present disclosure can be in the form of a capsule.

An exemplary dosage form or pharmaceutical composition of the present disclosure comprises 0.5 to 20 wt % of a pitolisant salt (e.g., pitolisant hydrochloride) and 2 to 50 wt % alkalinizing agent, where weight is based on the total weight of the dosage form or pharmaceutical composition. For example, an exemplary tablet of the present disclosure comprises (in weight): 0.5 to 20% of pitolisant salt such as hydrochloride; and 2 to 50% alkalinizing agent, with respect to the total weight of the tablet (e.g., coated tablet).

A pharmaceutical composition or dosage form of the present disclosure can comprise a coating, such as a film (sometimes referred to herein as a coating film). In embodiments, the dosage form or pharmaceutical composition is a tablet that comprises a core coated by a film, wherein said core comprises a pitolisant salt, such as pitolisant hydrochloride, and an alkalinizing agent. The core may further comprise one or more pharmaceutically acceptable excipients chosen from, e.g., microcrystalline cellulose, magnesium stearate, anhydrous colloidal silica, lactose, and croscarmellose sodium.

A coating film may comprise one or more ingredients including, for example, polyvinyl alcohol, titanium oxide, macrogol or talc. Suitable ready-to-use compositions for preparing a coating film (herein called “coating systems”) are commercially available, such as the Opadry® ambII coating system produced by Colorcon.

The coating film may be an anti-moisture barrier. It will be understood that an anti-moisture barrier is distinct from a delayed-release coating (sometimes referred to as an enteric coating). The anti-moisture barrier coating can be positioned around the core of a pharmaceutical composition or dosage form of the present disclosure, and optionally can be present in addition to a delayed-release (e.g., enteric) coating layer. For example, a dosage form or pharmaceutical composition of the present disclosure can comprise a coating film between the outer surface of the core and the inner surface of a delayed-release coating layer. The anti-moisture barrier may comprise any suitable material, such as material that prevents moisture from the environment readily entering the core of the dose form prior to its intended dissolution (e.g., upon administration). The anti-moisture barrier may comprise a polymer. Suitable polymers include polyvinyl alcohols and hypromellose (hydroxypropylmethylcellulose). An exemplary anti-moisture barrier material of the present disclosure is OPADRY® amb II.

Another exemplary pharmaceutical composition or dosage form of the present disclosure is a coated tablet, said coated tablet comprising: a core comprising 3 to 15 wt % of pitolisant hydrochloride; 20 to 50 wt % of alkalinizing agent; 35 to 50 wt % of one or more pharmaceutically acceptable excipients chosen from microcrystalline cellulose, magnesium stearate, anhydrous colloidal silica, and lactose; and a coating film, said coating film representing 2 to 5 wt %, with respect to the total weight of the coated tablet.

A dosage form or pharmaceutical composition of the present disclosure may be in the form of a gastro-resistant tablet or capsule.

An exemplary gastro-resistant tablet of the present disclosure comprises: a core comprising: 0.5 to 20 wt % of pitolisant hydrochloride; 2 to 20 wt % of alkalinizing agent; 40 to 60 wt % of pharmaceutically acceptable excipients chosen from microcrystalline cellulose, magnesium stearate, anhydrous colloidal silica, lactose, and croscarmellose sodium; a coating film comprising: 5 to 15 wt % of one or more gastro-resistant coating agents, with respect to the total weight of the coated tablet; and 2 to 5 wt % of pharmaceutically acceptable excipients chosen from coating systems and plasticizers; with respect to the total weight of the coated tablet.

As used herein “gastro-resistant coating agent” refers to a composition suitable for preparing a gastro-resistant film. Suitable ready-to-use compositions for preparing such gastro-resistant coating film are commercially available, such as the Acryl EZE® composition produced by Colorcon.

The dosage form or pharmaceutical composition can comprise a therapeutically effective amount of pitolisant salt. For example, the dosage form or pharmaceutical composition may comprise between about 1 mg and about 200 mg, e.g., between about 1 mg and about 100 mg, between about 1 mg and about 50 mg, between about 10 mg and about 25 mg, or between about 1 mg and about 10 mg, of pitolisant hydrochloride, e.g, about 3 mg, about 4 mg, about 5 mg, about 6 mg, about 8 mg, about 10 mg, about 12 mg, about 15 mg, about 20 mg, about 25 mg, about 30 mg, about 40 mg, or about 50 mg, of pitolisant hydrochloride.

The dosage form of pharmaceutical composition can comprise between about 1 mg and about 400 mg pitolisant or a pharmaceutically acceptable salt thereof (e.g., between about 1 mg and about 250 mg, or between about 20 mg and about 200 mg, e.g., about 10 mg, about 20 mg, about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, or about 300 mg). The dosage form or pharmaceutical composition can comprise between about 1 mg and about 80 mg pitolisant or a pharmaceutically acceptable salt thereof (e.g., between about 1 mg and about 70 mg, between about 1 mg and about 60 mg, between about 1 mg and about 50 mg, between about 1 mg and about 40 mg, between about 20 mg and about 40 mg, between about 40 mg and about 60 mg, or between about 50 mg and about 70 mg). For example, the dosage form or pharmaceutical composition can comprise about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, or about 75 mg of pitolisant or a pharmaceutically acceptable salt thereof.

It will be understood that the amount of pitolisant in the form of the free base in the dosage form or pharmaceutical composition will be slightly lower than the equivalent amount as mentioned above. For example, a dosage form or pharmaceutical composition disclosed herein comprising 5 mg of pitolisant hydrochloride will comprise about 4.45 mg of pitolisant (freebase). In another example, a dosage form or pharmaceutical composition disclosed herein comprising 20 mg of pitolisant hydrochloride comprises about 17.8 mg of pitolisant (freebase). In some embodiments, a dosage form or pharmaceutical composition disclosed herein comprises about 5 mg of pitolisant monohydrochloride, or about 4.45 mg pitolisant (freebase). In some embodiments, a dosage form or pharmaceutical composition disclosed herein comprises about 20 mg of pitolisant monohydrochloride, or about 17.8 mg pitolisant (freebase).

The dosage forms of the present disclosure can be tablets, caplets, capsules, suspensions, granules, powders, or the like. For example, the dosage form of said pharmaceutical composition is a tablet, more particularly a film-coated tablet.

A dosage form or pharmaceutical composition of the present disclosure can comprise a core, and optionally a coating surrounding the core. The core can comprise a combination of the present disclosure. According to an embodiment, the core comprises a combination of pitolisant hydrochloride and an alkalinizing agent.

In some embodiments, the dosage form or pharmaceutical composition is a coated tablet, and the active pharmaceutical ingredient (e.g., pitolisant HCl) is contained within the core of the tablet, and is not substantially present in the coating. In some embodiments, the dosage form or pharmaceutical composition is a coated tablet, and the alkalinizing agent is contained within the core of the tablet, and is not substantially present in the coating. In some embodiments, the dosage form or pharmaceutical composition is a coated tablet, and both the active pharmaceutical ingredient (e.g., pitolisant HCl) and alkalinizing agent are contained within the core of the tablet, and neither the active pharmaceutical ingredient nor the alkalinizing agent is substantially present in the coating.

The dosage forms or pharmaceutical compositions of the present disclosure can further comprise, in addition to the hydrochloride salt of the active pharmaceutical ingredient and the alkalinizing agent, one or more pharmaceutically acceptable excipients, such as diluents, dispersing agents, granulating agents, surface active agents, emulsifiers, disintegrating agents (sometimes referred to herein as disintegrants), binding agents (sometimes referred to herein as binders), preservatives, buffering agents, lubricating agents (sometimes referred to herein as lubricants), glidants, adjuvants, fillers, wetting agents, suspending agents, solvents, dispersion media, ion exchangers, salts, electrolytes, waxes, and/or oils, and the like.

The pharmaceutical composition or dosage form may comprise a pharmaceutically acceptable excipient disclosed herein, or a combination of pharmaceutically acceptable excipients disclosed herein.

For example, a dosage form or pharmaceutical composition of the present disclosure may comprise one or more, or all of, the following pharmaceutically acceptable excipients: colloidal silicon dioxide, crospovidone, magnesium stearate, microcrystalline cellulose, polyethylene glycol, polyvinyl alcohol, talc, and titanium dioxide.

Each pharmaceutically acceptable excipient can be present in the dosage form or pharmaceutical composition in any suitable amount. For example, a pharmaceutically acceptable excipient can be present in the dosage form or pharmaceutical composition in an amount of between about 0.01% and about 95% by weight of the dosage form or pharmaceutical composition, e.g., between about 0.1% and about 25%, between about 1% and about 10%, between about 15% and about 95%, between about 0.01% and about 2%, by weight of the dosage form or pharmaceutical composition.

Dosage forms and pharmaceutical compositions of the present disclosure may be administered orally, parenterally, by inhalation, topically, rectally, nasally, buccally, vaginally, or by implantation. In some embodiments, dosage forms and pharmaceutical compositions of the present disclosure are administered orally.

Preparation of the dosage forms or pharmaceutical composition of the present disclosure can include conventional methods, such as blending, filling, compressing (e.g., direct compression, compression of dry, wet or sintered granules), coating (e.g., coating in a spray process), extrusion, granulation (e.g., wet or dry granulation), pelleting (e.g., direct pelleting), binding, powder layering (e.g., onto active ingredient-free beads, or neutral cores or particles of pharmaceutically active agent), and rounding off.

### Methods of Treatment

The present disclosure also relates to a combination, dosage form or a pharmaceutical composition disclosed herein, for use for treating a disease or disorder, such as a disease or disorder disclosed herein.

The present disclosure also concerns a method for the treatment of a disease or disorder, comprising administering a combination, a dosage form, or a pharmaceutical composition disclosed herein to a subject in need thereof.

The disease or disorder can be a sleep disorder (e.g., excessive daytime sleepiness (EDS), cataplexy, narcolepsy, sleep apnea (e.g., obstructive sleep apnea), sleep induced apnea, diurnal somnolence), severe fatigue, idiopathic hypersomnia central nervous system disorder (e.g., epilepsy, Alzheimer's disease, Parkinson's disease, dementia (e.g., dementia with Lewy bodies and/or vascular dementia), attention disorders, wakefulness disorders, memorization disorders, cognitive deficits (e.g., in aged persons), psychiatric pathologies, depressive and asthenic states, vertigo, and motion sickness), obesity, psychosomatic disorders, respiratory disorders, allergic conditions, inflammatory conditions, cardiac conditions, gastrointestinal conditions, conditions of the urogenital system, conditions of the cutaneous system, stress, migraine, headache, pain, psychotropic disorders, asthma, bronchitis, rhinitis, tracheitis, gastric ulcers, duodenal ulcers, ulcerative colitis, Crohn's disease, irritable bowel syndrome (IBS), cystitis, metritis, urinary incontinence, fecal incontinence, urticaria, itching, arthritis, conjunctivitis, premenstrual syndrome, prostatic inflammations, genital disorders, rheumatic conditions, ocular conditions, sialorrhea, convulsion, depression, disorders of the hypothalamo-hypophyseal system, disorders of the cerebral circulation, and disorders of the immune system.

The disease or disorder can be a sleep disorder. For example, the disease or disorder can be excessive daytime sleepiness (EDS), cataplexy, narcolepsy, sleep apnea (e.g., obstructive sleep apnea), sleep induced apnea, diurnal somnolence, severe fatigue or idiopathic hypersomnia.

In some embodiments, the disease or disorder is excessive daytime sleepiness (EDS). In some embodiments, the disease or disorder is cataplexy. In a method disclosed herein, the subject to be treated may have narcolepsy, and/or may be an adult with narcolepsy. For example, the method of treating a disease or disorder may comprise administering to a subject in need thereof a combination or pharmaceutical composition of the present disclosure.

In some aspects, the present disclosure relates to a combination of a pitolisant salt (e.g., pitolisant hydrochloride) with an alkalinizing agent, or a dosage form or pharmaceutical composition disclosed herein, for use in the treatment of a disease or disorder (e.g., a disease or disorder disclosed herein), optionally, wherein the disease or disorder is excessive daytime sleepiness (EDS), cataplexy, narcolepsy, sleep apnea (e.g., obstructive sleep apnea), sleep induced apnea, diurnal somnolence, severe fatigue or idiopathic hypersomnia. The disease or disorder may be in a subject with narcolepsy (e.g., an adult subject with narcolepsy).

In some aspects, the present disclosure relates to the use of a combination of a pitolisant salt (e.g., pitolisant hydrochloride) with an alkalinizing agent, or a dosage form or pharmaceutical composition disclosed herein, for the manufacture of a medicament for the treatment of a disease or disorder (e.g., a disease or disorder disclosed herein), optionally, wherein the disease or disorder is excessive daytime sleepiness (EDS), cataplexy, narcolepsy, sleep apnea (e.g., obstructive sleep apnea), sleep induced apnea, diurnal somnolence, severe fatigue or idiopathic hypersomnia. The disease or disorder can be in a subject with narcolepsy (e.g., an adult subject with narcolepsy).

The present disclosure further relates to a method for the prevention of undesirable side effects associated with using antipsychotic or antidepressant agents (e.g., aripiprazole, clozapine, olanzapine, risperidone, quetiapine, sertindole, mirtazapine, amitriptyline, and paroxetine), comprising administering a combination, dosage form, or pharmaceutical composition of the present disclosure, to a subject in need thereof. Non-limiting examples of undesirable side effects associated with using antipsychotic or antidepressant agents includes weight gain, somnolence, and cognitive impairment.

The present disclosure further relates to a method for (i) inducing an extended state of wakefulness; (ii) improving cognitive processes; (iii) reducing food intake; and/or (iv) normalizing vestibular reflexes, comprising administering a combination, dosage form, or pharmaceutical composition disclosed herein, to a subject in need thereof.

The disease or disorder can be a depressive disorder, an anxiety disorder, chronic hives, insomnia, or itchiness.

The combination, dosage form, or pharmaceutical composition disclosed herein, may be administered once daily, twice daily, or more often. More than one dosage form can be administered at once to achieve a desired dose of the active ingredient. The combination, dosage form or pharmaceutical composition disclosed herein, may be taken with a frequency and in such an amount so that the total amount of active ingredient (e.g., pitolisant, in terms of freebase) administered is within the range of from about 5 mg to about 100 mg per day, e.g., about 5 mg to about 80 mg per day, e.g., about 5 mg to about 70 mg per day, e.g., about 10 mg to about 50 mg per day, e.g., about 15 mg to about 40 mg per day. The combination, or a dosage form or pharmaceutical compositions disclosed herein, may be taken with a frequency and in such an amount so that the total amount of active ingredient (e.g., pitolisant, in terms of freebase) administered is within the range of from about 17.8 mg to about 35.6 mg per day. For example, a subject may be administered orally two dosage forms once daily, where each dosage form comprises 4.45 mg active ingredient (e.g., pitolisant, in terms of freebase), to achieve a daily dose of 8.9 mg active ingredient (e.g., pitolisant, in terms of freebase). A subject may be administered orally one dosage form once daily, where the dosage form comprises 17.8 mg active ingredient (e.g., pitolisant, in terms of freebase), to achieve a daily dose of 17.8 mg active ingredient (e.g., pitolisant, in terms of freebase). A subject may be administered orally two dosage forms once daily, where each dosage form comprises 17.8 mg active ingredient (e.g., pitolisant, in terms of freebase), to achieve a daily dose of 35.6 mg active ingredient (e.g., pitolisant, in terms of freebase).

In some embodiments, the combination, dosage form or pharmaceutical composition disclosed herein, may be taken with a frequency and in such an amount so that the total amount of pitolisant (in terms of freebase) administered is within the range of from about 10 mg to about 50 mg per day, e.g., about 15 mg to about 40 mg per day. The combination, or a dosage form or pharmaceutical compositions disclosed herein, may be taken with a frequency and in such an amount so that the total amount of pitolisant (in terms of freebase) administered is within the range of from about 17.8 mg to about 35.6 mg per day. For example, a subject may be administered orally two dosage forms once daily, where each dosage form comprises 4.45 mg pitolisant (in terms of freebase), to achieve a daily dose of 8.9 mg pitolisant (in terms of freebase). A subject may be administered orally one dosage form once daily, where the dosage form comprises 17.8 mg pitolisant (in terms of freebase), to achieve a daily dose of 17.8 mg pitolisant (in terms of freebase). A subject may be administered orally two dosage forms once daily, where each dosage form comprises 17.8 mg pitolisant (in terms of freebase), to achieve a daily dose of 35.6 mg pitolisant (free base).

### Use for Improving Drug Absorption

The present disclosure also provides methods or uses for improving absorption of active ingredient (e.g., pitolisant or doxepin).

The present disclosure provides for improving absorption of an active ingredient, said use comprising administering said active ingredient in the form of a salt together with an alkalinizing agent, such as defined above. For example, a method or use of the present disclosure for improving absorption of active ingredient (e.g., pitolisant or doxepin) can comprise administering the active ingredient (optionally, in the form of a salt) together with an alkalinizing agent, such as an alkalinizing agent of the present disclosure. The administering can be orally administering.

A method or use of the present disclosure for improving absorption of active ingredient (e.g., pitolisant or doxepin) can comprise administering a combination, dosage form, or pharmaceutical composition of the present disclosure. The administering can be orally administering.

Improving absorption using a method of the present disclosure can comprise accelerating the absorption of the active ingredient within the patient's body (e.g., following oral administration, e.g., absorption from the gastrointestinal tract into the bloodstream), e.g., as determined by a shortening of Tₘₐₓ. Improving absorption using a method of the present disclosure can comprise increasing the amount of active ingredient absorbed (e.g., following oral administration, e.g., absorption from the gastrointestinal tract into the bloodstream), e.g., as determined by a higher Cₘₐₓ.

In some aspects, the present disclosure concerns an accelerated absorption formulation comprising an active pharmaceutical ingredient (optionally, in the form of a salt) combined with an alkalinizing agent.

### Definitions

The articles “a” and “an” are used herein to refer to one or more than one (i.e., to at least one) of the grammatical object of the article. By way of example, “an element” means one element or more than one element.

The term “about” when referring to a measurable value such as an amount, a temporal duration, and the like, is meant to encompass variations of 20% or less, or in some instances ±15% or less, or in some instances ±10% or less, or in some instances ±5% or less, or in some instances 10% or less, or in some instances ±0.1% or less, from the specified value, as such variations are appropriate.

The phrase “and/or” as used herein should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “administer,” “administering,” or “administration,” as used herein refer to implanting, absorbing, ingesting, injecting, inhaling, or otherwise introducing a compound, dosage form, or pharmaceutical composition.

The terms “comprise,” “comprises,” and “comprising” are used herein in a non-exclusive sense, except where the context requires otherwise. Likewise, the term “include” and its grammatical variants are intended to be non-limiting, such that recitation of items in a list is not to the exclusion of other like items that can be substituted or added to the listed items.

The term “effective amount” or a “therapeutically effective amount” as used herein refers to an amount of a compound, or a pharmaceutical composition, described herein, which is sufficient to achieve a desired result under the conditions of administration. For example, an effective amount of a compound, combination, dosage form, or pharmaceutical composition disclosed herein for treating excessive sleep disorder (EDS), e.g., in a subject with narcolepsy, is an amount that can reduce the effects of the EDS, and/or reduce or eliminate the severity of a symptom associated with the EDS. A skilled clinician can determine appropriate dosing based on a variety of considerations including the severity of the disease, the subject's age, weight, general health and other considerations.

The term “pharmaceutically acceptable excipient” as used herein refers to a nontoxic material that may be formulated with a compound or combination disclosed herein (e.g., pitolisant or doxepin in combination with an alkalinizing agent) to provide a dosage form or pharmaceutical composition. Preferably, the pharmaceutically acceptable excipient is inert and does not interfere with the pharmacological activity of a compound (e.g., active pharmaceutical ingredient) which it is formulated with. Pharmaceutically acceptable excipients useful in the manufacture of the dosage forms and pharmaceutical compositions disclosed herein are any of those well known in the art, and include without limitation, diluents, dispersing agents, granulating agents, surface active agents, emulsifiers, disintegrating agents (sometimes referred to herein as disintegrants), binding agents (sometimes referred to herein as binders), preservatives, buffering agents (sometimes referred to herein as buffers), lubricating agents (sometimes referred to herein as lubricants), glidants, adjuvants, fillers, wetting agents, suspending agents, solvents, dispersion media, ion exchangers, salts, electrolytes, waxes, and/or oils, and the like.

For example, a pharmaceutically acceptable excipient may be alumina, a phosphate (e.g., calcium phosphate, dicalcium phosphate, tricalcium phosphate, disodium hydrogen phosphate, potassium hydrogen phosphate), a sulfate (e.g., calcium sulfate), a cellulose, kaolin, bentonite, lactose, mannitol, sorbitol, sucrose, inositol, compressible sugar, trehalose, xylitol, acacia, gelatin, glucose, maltodextrin, starch (e.g., corn starch, potato starch), sodium starch glycolate, starch derivatives, an amino acid, polyvinylpyrrolidone (PVP, povidone) (e.g., crosslinked PVP, crospovidone), polyvinyl alcohol, tragacanth, polyethylene glycol, mineral clay powders, croscarmellose, poloxamer, fatty acids or salts thereof (e.g., lauric acid, sodium lauryl sulfate, stearic acid, calcium stearate, magnesium stearate, aluminum stearate, oleic acid), hydrogenated vegetable oils, talc, titanium dioxide, glyceryl behenate, silicon dioxide (e.g., colloidal silicon dioxide), a silicate salt (e.g., magnesium trisilicate), lecithin, serum protein (e.g., human serum albumin), sorbic acid, potassium sorbate, a metal cation salt (e.g., a sodium salt, such as sodium chloride, a potassium salt, such as potassium chloride, a magnesium salt, such as or magnesium chloride, a zinc salt, such as zinc chloride), water, dimethylacetamide, protamine sulfate, wool fat, ethylenediaminetetraacetic acid (EDTA), a cyclodextrin (e.g., CAPTISOL*), a polysorbate (e.g., TWEEN®, e.g., TWEEN® 20 or TWEEN® 80), and combinations thereof.

The term “pharmaceutically acceptable salt” as used herein refers to salts of a compound prepared with relatively nontoxic acids or bases, depending on the particular substituents found on the respective compound. When compounds contain relatively basic functionalities, acid addition salts can be obtained by contacting the neutral form of such compounds with the desired acid, either neat or in a suitable solvent (e.g., an inert solvent). Examples of pharmaceutically acceptable acid addition salts include those derived from inorganic acids like hydrochloric acid, hydrobromic acid, nitric acid, carbonic acid, monohydrogen carbonic acid, phosphoric acid, monohydrogen phosphoric acid, dihydrogen phosphoric acid, sulfuric acid, monohydrogen sulfuric acid, hydriodic acid, or phosphorous acids and the like, as well as the salts derived from organic acids like acetic acid, propionic acid, isobutyric acid, maleic acid, malonic acid, benzoic acid, succinic acid, suberic acid, fumaric acid, lactic acid, mandelic acid, pamoic acid, phthalic acid, benzenesulfonic acid, p-toluene sulfonic acid, citric acid, tartaric acid, methanesulfonic acid, oxalic acid, and the like. Also included are salts of amino acids such as arginine salt and the like, and salts of organic acids like glucuronic or galacturonic acids and the like. Other pharmaceutically acceptable salts known to those of skill in the art are suitable for pharmaceutical compositions relating to the present disclosure.

The term “solvate” as used herein refers to forms of a compound that are associated with a solvent, usually by a solvolysis reaction. This physical association may include hydrogen bonding. Conventional solvents include water, methanol, ethanol, acetic acid, dimethyl sulfoxide (DMSO), tetrahydrofuran (THF), diethyl ether, and the like. Compounds of the present disclosure may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” as used herein refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “subject” as used herein refers to any animal, such as any mammal, including but not limited to, humans, non-human primates, rodents, dogs, and the like. Non-human primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, feline species (e.g., domestic cat), canine species (e.g., dog, fox, wolf), avian species, and fish. In some embodiments, the subject is a mammal (e.g., a human, a rat, or a mouse). The subject can be male or female. The subject may be of any age, including an elderly human subject (e.g., 65 years or older), a human subject that is not elderly (e.g., less than 65 years old), or a human pediatric subject (e.g., less than 18 years old). In preferred aspects, the subject is a human.

As used herein, the terms “treat,” “treatment,” “treating,” or grammatically related terms, refer to a method of reducing the effects of a disease or disorder. As is readily appreciated in the art, full eradication of the disease, disorder, or symptoms thereof is preferred but not a requirement for treatment. Desirable effects of treatment include, but are not limited to, preventing occurrence or recurrence of the disease or disorder, alleviation of symptoms, diminishment of any direct or indirect pathological consequences of the disease or disorder, or other improvement of any sign, symptom, or consequence of the disease or disorder, such as prolonged survival, less morbidity, and/or a lessening of side effects.

Throughout this disclosure, various embodiments can be presented in a range format (e.g., from X to Y). It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range, such as from 1 to 6, should be considered to have specifically disclosed subranges such as from 1 to 5, from 1 to 4, from 1 to 3, from 2 to 6, from 2 to 4, from 3 to 6, etc., as well as individual numbers within that range, e.g., 1, 2, 2.8, 3, 3.6, 4, 5, 5.4, and 6. As another example, a range such as 95-99% includes 95%, 96%, 97%, 98%, or 99% and all subranges such as 96-99%, 96-98%, 96-97%, 97-99%, 97-98%, etc. This applies regardless of the breadth of the range

All publications (e.g., scientific journal articles, patent publications, and the like) cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

Compounds (e.g., pharmaceutically active agents) disclosed herein may also comprise one or more isotopic substitutions. For example, hydrogen (H) may be in any isotopic form, including ¹H, ²H (D or deuterium), ³H (T or tritium); carbon (C) may be in any isotopic form, including ¹²C, ¹³C, and ¹⁴C; oxygen (O) may be in any isotopic form, including ¹⁶O and ¹⁸O; nitrogen (N) may be in any isotopic form, including ¹⁴N and ¹⁵N; and chlorine (Cl) may be in any isotopic form, including ³⁵Cl and ³⁷Cl.

Various embodiments of the combinations, pharmaceutical compositions, and methods herein are described in further detail below, and additional definitions may be provided throughout the specification.

### EXAMPLES

### Example 1. Exemplary Formulations Comprising Pitolisant Hydrochloride

Exemplary formulations comprising pitolisant hydrochloride (40 mg), with an alkalinizing agent at low and high dose, and pharmaceutically acceptable excipients were prepared: Treatment A: 30 mg magnesium carbonate; Treatment B: 7.5 mg magnesium carbonate; Treatment C: 30 mg potassium bicarbonate; Treatment D: 30 mg sodium carbonate.

Each formulation was provided as a tablet coated with a gastro-resistant film, the details of which are provided in Table 1 below.

TABLE 1
Exemplary formulations.

| | Unit | Unit | Unit | Unit |
|---|---|---|---|---|
| | formula | formula | formula | formula |
| Raw material | (mg/unit) | (mg/unit) | (mg/unit) | (mg/unit) |
| Formulation | Treatment A | Treatment B | Treatment C | Treatment D |
| Pitolisant HCl | 40.00^{a} | 40.00^{a} | 40.00^{a} | 40.00^{a} |
| Magnesium carbonate | 30.00 | 7.50 | / | |
| Potassium bicarbonate | / | / | 30.00 | 1 |
| Sodium carbonate | / | / | / | 30.00 |
| Lactose | 107.17 | 122.17 | 107.17 | 107.17 |
| Microcrystalline cellulose | 53.58 | 61.08 | 53.58 | 53.58 |
| Croscarmellose sodium | 11.25 | 11.25 | 11.25 | 11.25 |
| Anhydrous colloidal silica | 2.00 | 2.00 | 2.00 | 2.00 |
| Magnesium stearate | 6.00 | 6.00 | 6.00 | 6.00 |
| TOTAL | 250.0 | 250.0 | 250.0 | 250.0 |
| Coating | | | | |
| Opadry amb II¹ (clear, 88A190022) | 7.5 | 7.5 | 7.5 | 7.5 |
| Acryl Eze² 93A19346 clear | 38.625 | 38.625 | 38.625 | 38.625 |
| PEG 8000³ | 3.09 | 3.09 | 3.09 | 3.09 |
| TOTAL | 299.215 | 299.215 | 299.215 | 299.215 |

^{a}Salt/Base ratio = 1.1234 - corresponding to 35.6 mg of pitolisant base. Pitolisant doses are expressed as pitolisant-HCl dose in this protocol. 40 mg pitolisant-HCl dose corresponding to 35.6 mg pitolisant base.
¹15% suspension, weight gain = 3%
²20% suspension, weight gain = 15%
³PEG at 8% of Acryl Eze

### Example 2: Clinical Study Using Exemplary Formulations of the Present Disclosure

Exemplary formulations (Treatments A and B) were assayed in an open label, single-center, cross-over randomized, relative bioavailability study comparing exemplary pitolisant HCl tablet formulations versus a reference pitolisant HCl (WAKIX®) tablet formulation after single oral administrations in healthy male subjects. Sixteen (16) healthy normal weight male subject were enrolled in order to get at least twelve (12) complete evaluable subjects.

The bioequivalence of exemplary formulations A and B (Treatments A and B) as disclosed in Example 1 comprising pitolisant hydrochloride (40 mg) with an alkalinizing agent at high and low doses was determined and compared with the reference formulation, as outlined in Table 2:

TABLE 2
Statistical results of bioequivalence assessment

| | | | Reference | | |
|---|---|---|---|---|---|
| | | | formulation | Test | |
| Analysis | Parameter | W-CV (%) | GM | Treatment | GM |
| Bioequivalence | Cₘₐₓ (ng/mL) | 26.7 | 36.45 | A | 34.74 |
| | | | | B | 42.48 |
| | AUC₀₋ₜ (h · ng/mL) | 21.7 | 441.80 | A | 505.83 |
| | | | | B | 523.50 |
| | AUC_{0-inf} (h · ng/ml) | 21.7 | 454.84 | A | 520.61 |
| | | | | B | 543.26 |

GM: Geometric mean - GMR: Geometric mean ratio - CI: Confidence Interval - BE: Bioequivalence - W-CV: Within subject CV(%)
Reference: Two 20 mg Wakix ® tablets
The data above show an increase of pitolisant bioavailability of ~15 to 20% (AUCs and/or Cₘₐₓ) with magnesium carbonate
(Low 7.5 mg dose (Treatment B) and high dose 30 mg (Treatment A)).

### Example 3. Apparent Permeability Over Time of Exemplary Pitolisant HCl Combinations

Caco-2 permeability assay uses an established method that measures the rate of flux of a compound across polarized Caco-2 cell monolayers and from which the data generated can be used to predict in vivo absorption of drugs.

The Caco-2 cell line is derived from a human colon carcinoma. The cells have characteristics that resemble intestinal epithelial cells such as the formation of a polarized monolayer, well-defined brush border on the apical surface and intercellular junctions. The cells were grown on a semipermeable membrane in a Transwell™ system plate and form a confluent polarized monolayer over 18-22 days. On day 18-22, the test compound is added to the apical side of the cell monolayer and the flux of the compound across the monolayer, into the basolateral receiver compartment, is monitored up to a 2-hour incubation period (various intermediate time points).

The permeability coefficient (Papp) is calculated from the following equation:

Papp=dQ/dtC0×A

Where Papp is the apparent permeability coefficient (cm/s×10−6), dQ/dt is the rate of permeation of the drug across the cells (pmol/sec), CO is the donor concentration at time zero (pmol/mL) and A is the area of the cell monolayer (cm2).

The apparent permeabilities of exemplary formulations comprising pitolisant hydrochloride (40 mg) with different alkalinizing agents were determined and compared with a control (HBSS buffer, pH 7), as outlined in Table 3:

TABLE 3
Exemplary combinations of pitolisant hydrochloride
for apparent permeability study.

| Formulation | Components/pH |
|---|---|
| 2A | pitolisant hydrochloride (40 mg), potassium |
| | bicarbonate (30 mg) pH 8.0 |
| 2B | pitolisant hydrochloride (40 mg), potassium |
| | bicarbonate (15 mg) pH 7.9 |
| 2C | pitolisant hydrochloride (40 mg), potassium |
| | bicarbonate (7.5 mg) pH 7.5 |
| 2D | pitolisant hydrochloride (40 mg), magnesium |
| | oxide (30 mg) pH 11.0 |
| 2E | pitolisant hydrochloride (40 mg), magnesium |
| | oxide (15 mg) pH 10.6 |
| 2F | pitolisant hydrochloride (40 mg), magnesium |
| | oxide (7.5 mg) pH 10.6 |
| control | pitolisant hydrochloride (40 mg) with HBSS |
| | buffer pH 7 |

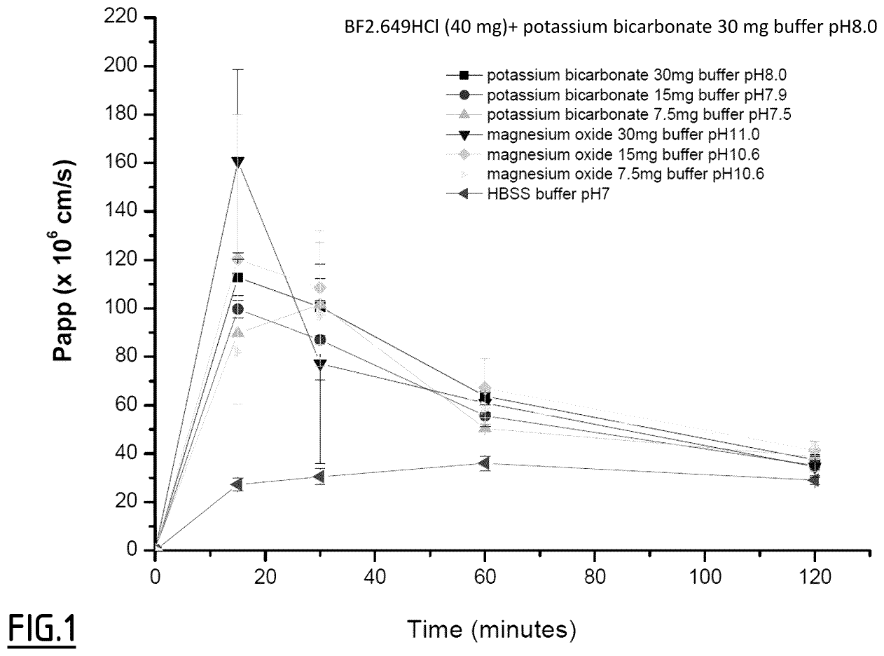

The apparent permeabilities of the formulations are provided in FIG. 1, for pitolisant hydrochloride concentration of 540 μM corresponding to a dose of 40 mg pitolisant HCl tablet dissolved with a standardized volume of 250 ml of drinking water
In standard permeability assay conditions (control): pitolisant HCl dissolved in HBSS buffer pH 7;
Pitolisant solubilized in water+potassium carbonate (7.5 or 15 or 30 mg); and
Pitolisant solubilized in water+magnesium oxide (7.5 or 15 or 30 mg).

The results show that the combinations of pitolisant hydrochloride with an alkalinizing agent result in a relatively an early peak (i.e., shorter Tₘₐₓ), together an increased permeability, compared to control (pitolisant hydrochloride with buffer).

### Example 4. Bioavailability of Exemplary Pitolisant HCl Combinations

The bioavailability of pitolisant hydrochloride was assessed at a concentration of 540 μM corresponding to a dose of 40 mg pitolisant HCl tablet solubilized with a standardized volume of 250 ml of drinking water, either in standard permeability assay conditions (pitolisant HCl dissolved in HBSS buffer pH 7) or with an alkalinizing agent with a set pH.

The bioavailability of exemplary combinations of pitolisant hydrochloride with alkalinizing agent was determined by measuring the area under the curve (AUC₀₋₁₂₀ min, expressed as % over control), as shown in Table 4.

TABLE 4
Exemplary combinations of pitolisant hydrochloride
for bioavailability study.

| Formulation | Components/pH |
|---|---|
| 1 (control) | (pitolisant hydrochloride 40 mg/HBSS buffer pH 7) |
| 2 | pitolisant hydrochloride 40 mg pH 8 |
| 3 | pitolisant hydrochloride 40 mg/potassium |
| | bicarbonate 7.5 mg pH 7.5 |
| 4 | pitolisant hydrochloride 40 mg/sodium carbonate |
| | 7.5 mg pH 8.6 |
| 5 | pitolisant hydrochloride 40 mg/potassium |
| | bicarbonate 15 mg pH 7.9 |
| 6 | pitolisant hydrochloride 40 mg/sodium carbonate |
| | 30 mg pH 9.5 |
| 7 | pitolisant hydrochloride 40 mg/magnesium oxide |
| | 30 mg pH 11 |
| 8 | pitolisant hydrochloride 40 mg/magnesium |
| | carbonate 30 mg HBSS pH 9.8 |
| 9 | pitolisant hydrochloride 40 mg/magnesium |
| | carbonate 15 mg HBSS pH 9.9 |
| 10 | pitolisant hydrochloride 40 mg/potassium |
| | bicarbonate 30 mg pH 8 |
| 11 | pitolisant hydrochloride 40 mg/magnesium oxide |
| | 7.5 mg pH 9.8 |
| 12 | pitolisant hydrochloride 40 mg/magnesium |
| | carbonate 7.5 mg HBSS pH 9.7 |
| 13 | pitolisant hydrochloride 40 mg/magnesium |
| | oxide 15 mg pH 10.6 |

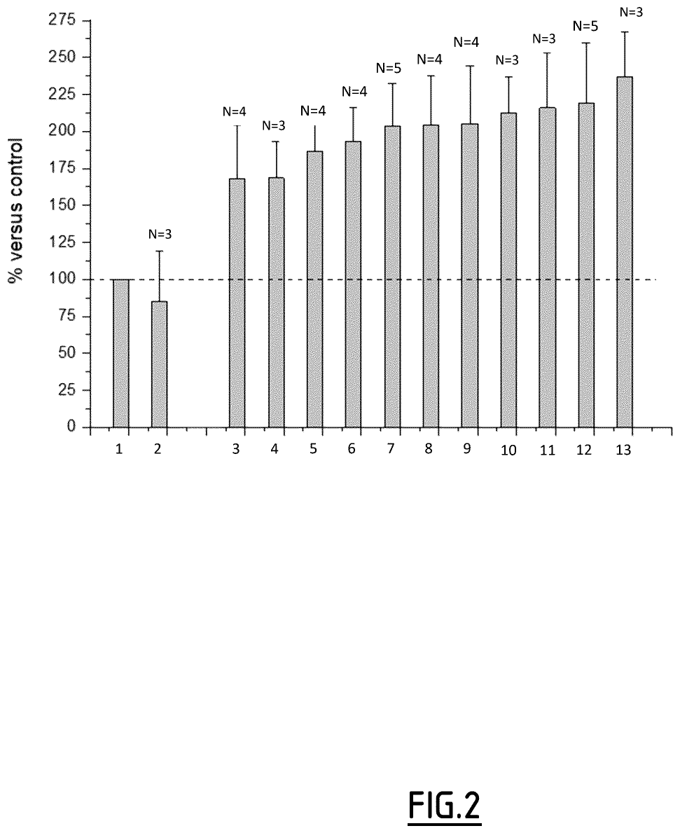

The results of the bioavailability measurement are provided in FIG. 2. The results show that the combinations of pitolisant hydrochloride with an alkalinizing agent led to a higher bioavailability compared to control (pitolisant hydrochloride with buffer).

### Example 5. Apparent Permeability Over Time of an Exemplary Doxepin HCl Combination

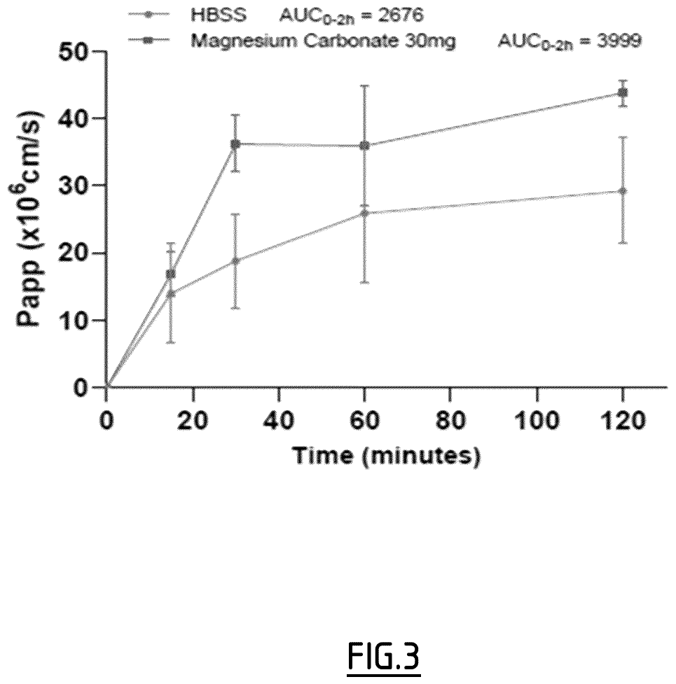

Apparent permeability was assessed using the Caco-2 permeability assay as disclosed above. The apparent permeability of an exemplary combination of doxepin hydrochloride (10 mg) with magnesium carbonate (30 mg), pH 7.5, was compared with control (doxepin hydrochloride 10 mg/HBSS. The results are provided in FIG. 3 which illustrates a combination comprising doxepin hydrochloride at a concentration of 143 μM corresponding to a dose of 10 mg doxepin HCl tablet dissolved with a standardized volume of 250 ml of drinking water:
In standard permeability assay conditions (doxepin HCl dissolved in HBSS buffer pH 7), and
Doxepin solubilized in water+magnesium carbonate 30 mg

FIG. 3 shows that the combination of doxepin hydrochloride with an alkalinizing agent led to a higher bioavailability (+50% over 2 hours) compared to doxepin hydrochloride with buffer (Hanks' balanced salt solution, HBSS).

1. A combination of a salt of pitolisant with an alkalinizing agent.

2. The combination of claim 1, wherein the salt of pitolisant is pitolisant hydrochloride.

3. The combination of claim 1, wherein the alkalinizing agent comprises one or more of the following: tetraborate, hydroxide, oxide, carbonate, citrate, bicarbonate, acetate, lactate, arginine, lysine, choline, meglumine, tromethamine, potassium, magnesium, calcium, or sodium salts.

4. The combination of claim 1, wherein the alkalinizing agent is magnesium carbonate or sodium carbonate.

5. The combination of claim 1, wherein the weight ratio (alkalinizing agent/alkalinizing agent+pitolisant salt) is between 5 and 99%.

6. A method of treating or preventing a disease or disorder chosen from the group consisting of excessive daytime sleepiness (EDS), cataplexy, narcolepsy, sleep apnea, sleep induced apnea, diurnal somnolence, severe fatigue, and idiopathic hypersomnia, comprising administering to a subject in need thereof a combination of claim 1.

7. A method of accelerating and/or increasing the absorption of pitolisant in a subject, comprising administering to a subject in need thereof a combination of claim 1.

8. A pharmaceutical composition comprising a combination of claim 1, and optionally one or more pharmaceutically acceptable excipients.

9. The pharmaceutical composition of claim 8, wherein the composition is in the form of a tablet that comprises (in weight):
0.5 to 20 wt % pitolisant hydrochloride; and
2 to 50 wt % alkalinizing agent;
with respect to the total weight of the tablet.

10. The pharmaceutical composition of claim 8, wherein the tablet comprises a core coated by a film, and wherein the core comprises pitolisant hydrochloride and the alkalinizing agent.

11. The pharmaceutical composition to of claim 10, wherein the core further comprises one or more pharmaceutically acceptable excipients.

12. The pharmaceutical composition of claim 8, wherein the composition is in the form of a coated tablet comprising:
a core, wherein the core comprises:
3 to 15 wt % pitolisant hydrochloride;
20 to 50 wt % alkalinizing agent; and
35 to 50 wt % pharmaceutically acceptable excipient selected from the group consisting of microcrystalline cellulose, magnesium stearate, anhydrous colloidal silica, and lactose; and
a film coating on the core wherein the film coating is present in an amount of 2 to 5 wt %,
wherein all weights are with respect to the total weight of the coated tablet.

13. The pharmaceutical composition of claim 8, wherein the composition is in the form of a gastro-resistant tablet comprising:
a core, wherein the core comprises:
0.5 to 20 wt % pitolisant hydrochloride;
2 to 20 wt % alkalinizing agent;
40 to 60 wt % pharmaceutically acceptable excipient selected from the group consisting of microcrystalline cellulose, magnesium stearate, anhydrous colloidal silica, lactose, and croscarmellose sodium; and
a film coating on the core, wherein the film coating comprises:
5 to 15 wt % of one or more gastro-resistant coating agents, and
2 to 5 wt % pharmaceutically acceptable excipient selected from the group consisting of coating systems and plasticizers;
wherein all weights are with respect to the total weight of the gastro-resistant tablet.

14. An accelerated absorption formulation comprising an active pharmaceutical ingredient combined with an alkalinizing agent, wherein the active pharmaceutical ingredient comprises doxepin or a salt thereof, or pitolisant or a salt thereof.

15. A method for accelerating the absorption of an active pharmaceutical ingredient, comprising administering the active pharmaceutical ingredient to a subject together with an alkalinizing agent.

16. The accelerated absorption formulation of claim 14, wherein the active pharmaceutical ingredient is doxepin hydrochloride, or pitolisant hydrochloride.

17. The method of claim 15, wherein the active pharmaceutical ingredient comprises doxepin or a salt thereof, or pitolisant or a salt thereof.

18. The method of claim 15, wherein the alkalinizing agent is selected from the group consisting of tetraborate, hydroxide, oxide, carbonate, citrate, bicarbonate, acetate, lactate, arginine, lysine, choline, meglumine, tromethamine, potassium, magnesium, calcium, and sodium salts.

19. The combination of claim 1, wherein the alkalinizing agent is magnesium carbonate.

20. The method of claim 15, wherein the active pharmaceutical ingredient comprises a salt.
